(19) United States
(12) Patent Application Publication
(10) Pub. No.: US 2026/0128159 A1
(43) Pub. Date: May 7, 2026

(54) SYSTEM AND METHOD FOR AUTOMATED ANNOTATION OF VESSEL DIAGRAMS IN MEDICAL IMAGING PICTURE ARCHIVING AND COMMUNICATION SYSTEMS

(71) Applicant: FUJIFILM Healthcare Americas Corporation, Lexington, MA (US)

(72) Inventor: Pierre Eugene Fuller, Jacksonville, FL (US)

(73) Assignee: FUJIFILM Healthcare Americas Corporation, Lexington, MA (US)

(21) Appl. No.: 18/935,133

(22) Filed: Nov. 1, 2024

(51) Int. Cl. G16H 30/40; G16H 30/20

(52) U.S. Cl. CPC G16H 30/40; G16H 30/20

(57) ABSTRACT

A method, system and computer program product for automated annotation of vessel diagrams within a PACS, the PACS comprising medical images that include associated DICOM data. The method includes selecting a first imaging study corresponding to at least one anatomical structure; automatically identifying at least one vessel diagram corresponding to the at least one anatomical structure of the first imaging study based on the DICOM data associated with the first imaging study; selecting a first vessel diagram of the automatically identified at least one vessel diagram; displaying the first vessel diagram with a set of predefined annotation tools; determining if at least one characteristic of the at least one anatomical structure meets pre-determined criteria based on the DICOM data; when the at least one characteristic meets the pre-determined criteria, automatically incorporating at least one annotation with the first vessel diagram using at least one of the predefined annotation tools.

### TECHNICAL FIELD

The present invention relates to the field of medical picture archiving and communication systems (PACS); and more particularly relates to a system and method for automated annotation of vessel diagrams within a PACS.

### BACKGROUND OF THE INVENTION

In the field of medical imaging, the use of PACS has revolutionized the way medical professional's store, access, and analyze patient images. For example, PACS allows healthcare professionals to quickly access and conduct a detailed review of a plurality of medical images in digital format, which can lead to faster and more accurate diagnoses and treatment planning. Particularly, medical images in digital imaging and communications in medicine (DICOM) format that include information on vascular stenosis and stent placements play a crucial role for medical professionals in making diagnostic and therapeutic decisions.

In conventional technology, medical professionals manually annotate medical images with blood vessels by marking the locations of stenosis and stent placements. This method allows for the visual comprehension of diagnostic information of patients. However, several challenges exist with this approach. Manual annotation is time-consuming and labor-intensive, and there is a risk of human error. Additionally, the creation of detailed reports that include precise information on lesions and measurements of blood vessels requires an extensive amount of work, which can hinder the efficient execution of medical tasks.

Accordingly, there exists a need for an improved method and system within PACS that allow for the efficient creation of detailed reports that include annotated vessel diagrams and precise information on lesions and measurements of blood vessels.

### SUMMARY OF THE INVENTION

It is, therefore, an aspect of the present invention to provide a computer-implemented method for automated annotation of vessel diagrams within a PACS, the PACS comprising at least one imaging study including at least one medical image captured by a medical imaging apparatus, wherein the at least one medical image includes associated DICOM data.

The method includes the steps of selecting a first imaging study in a worklist graphical user interface (GUI) in response to a first user input, wherein the first imaging study corresponds to at least one anatomical structure; automatically identifying, in a clinical reporting application (CRA) GUI, at least one vessel diagram corresponding to the at least one anatomical structure of the first imaging study based on the DICOM data associated with the first imaging study; selecting a first vessel diagram of the automatically identified at least one vessel diagram in response to a second user input in the CRA GUI; displaying, in the CRA GUI, the first vessel diagram with a set of predefined annotation tools; determining if at least one characteristic of the at least one anatomical structure meets pre-determined criteria based on the DICOM data; when the at least one characteristic of the at least one anatomical structure meets the pre-determined criteria, automatically incorporating at least one annotation with the first vessel diagram using at least one of the predefined annotation tools; and storing, in a memory of the PACS, the first vessel diagram with the at least one annotation.

Another aspect of the present invention a computer system for automated annotation of vessel diagrams within a PACS, the PACS comprising at least one imaging study including at least one medical image captured by a medical imaging apparatus, wherein the at least one medical image includes associated DICOM data. The computer system includes one or more computer processors; one or more non-transient, computer-readable storage media; and program instructions stored on the one or more non-transient, computer-readable storage media for execution by at least one of the one or more computer processors.

The program instructions include program instructions to select a first imaging study in a worklist GUI in response to a first user input, wherein the first imaging study corresponds to at least one anatomical structure; automatically identify, in a CRA GUI, at least one vessel diagram corresponding to the at least one anatomical structure of the first imaging study based on the DICOM data associated with the first imaging study; select a first vessel diagram of the automatically identified at least one vessel diagram in response to a second user input in the CRA GUI; display, in the CRA GUI, the first vessel diagram with a set of predefined annotation tools; determine if at least one characteristic of the at least one anatomical structure meets pre-determined criteria based on the DICOM data; when the at least one characteristic of the at least one anatomical structure meets the pre-determined criteria, automatically incorporate at least one annotation with the first vessel diagram using at least one of the predefined annotation tools; and store, in a memory of the PACS, the first vessel diagram with the at least one annotation.

Another aspect of the present invention provides a computer program product for automated annotation of vessel diagrams within a PACS, the PACS comprising at least one imaging study including the at least one medical image captured by a medical imaging apparatus, wherein the at least one medical image includes associated DICOM data. The computer program product comprising a non-transient computer readable storage medium having program code embodied therewith.

The program code being executable by a processor to select a first imaging study in a worklist graphical user interface (GUI) in response to a first user input, wherein the first imaging study corresponds to at least one anatomical structure; automatically identify, in a CRA GUI, at least one vessel diagram corresponding to the at least one anatomical structure of the first imaging study based on the DICOM data associated with the first imaging study; select a first vessel diagram of the automatically identified at least one vessel diagram in response to a second user input in the CRA GUI; display, in the CRA GUI, the first vessel diagram with a set of predefined annotation tools; determine if at least one characteristic of the at least one anatomical structure meets pre-determined criteria based on the DICOM data; when the at least one characteristic of the at least one anatomical structure meets the pre-determined criteria, automatically incorporate at least one annotation with the first vessel diagram using at least one of the predefined annotation tools; and store, in a memory of the PACS, the first vessel diagram with the at least one annotation.

Additional aspects, advantages and novel features of the present invention will be set forth in part in the description, which follows and will in part become apparent to those in the practice of the invention, when considered with the attached figures.

### BRIEF DESCRIPTION OF THE DRAWINGS

The present invention will now be described, by way of example, with reference to the accompanying drawings, in which:

FIG. 1 is a block diagram illustrating an information processing environment for anonymous and secure sharing of medical images within a PACS in accordance with certain embodiments of the present invention;

FIG. 2 is an exemplary screen shot showing a worklist GUI and a viewer GUI in accordance with certain embodiments of the present invention;

FIG. 3 is a flow chart depicting operational steps for automated annotation of vessel diagrams within a PACS, in accordance with certain embodiments;

FIG. 4 is a block diagram illustrating one or more vessel diagrams, in accordance with some embodiments;

FIGS. 5A-5B are exemplary screen shots showing annotated vessel diagrams, in accordance with some embodiments;

FIGS. 6A-6E are block diagrams illustrating logic involved in automating annotations in vessel diagrams, in accordance with some embodiments;

FIGS. 7A-7B are depictions illustrating exemplary operational steps for automated annotation of vessel diagrams within a PACS, in accordance with some embodiments;

FIGS. 8 and 8A-8D illustrate an exemplary screen shot showing a first vessel diagram with annotations integrated in to a generated clinical report, in accordance with some embodiments; and

FIG. 9 depicts a block diagram of an exemplary computing device that may be used to execute the processes and methods disclosed herein.

Corresponding reference characters indicate corresponding parts throughout the several views. The exemplifications set out herein illustrate currently preferred embodiments of the invention, and such exemplifications are not to be construed as limiting the scope of the invention in any manner.

### DETAILED DESCRIPTION OF THE INVENTION

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer-readable medium(s) having computer-readable program code/instructions embodied thereon.

Any combination of computer-readable media may be utilized. Computer-readable media may be a computer-readable signal medium or a computer-readable storage medium. A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of a computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain, or store a program for use by or in connection with an instruction execution system, apparatus, or device.

Program code embodied on a computer-readable medium may be transmitted using any appropriate medium, including but not limited to wireless, wireline, optical fiber cable, radio frequency (RF), or any suitable combination of the foregoing.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on a user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable medium that can direct a computer, other programmable data processing apparatus, or other devices to function in a particular manner, such that the instructions stored in the computer-readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer-implemented process such that the instructions which execute on the computer or other programmable apparatus provide processes for implementing the functions/acts specified in the flowchart and/or block diagram block or blocks.

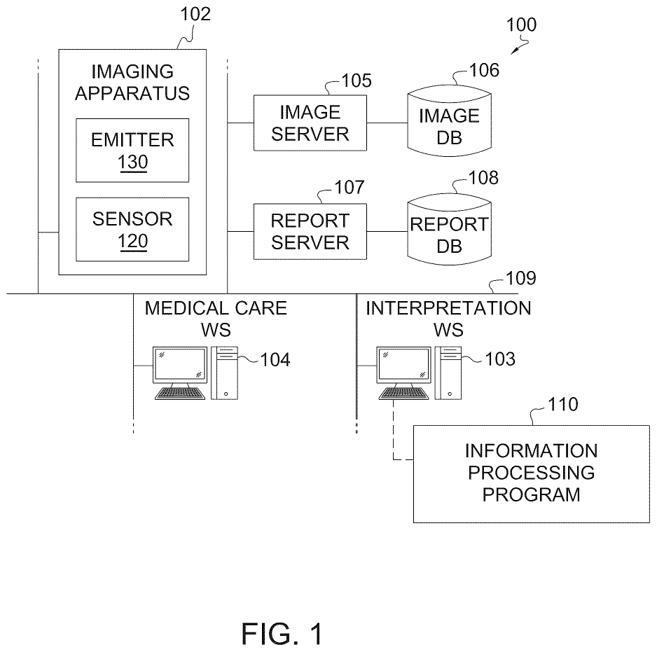

With initial reference to FIG. 1, a block diagram illustrating an information processing environment, generally 100, for capturing, storing, and/or analyzing medical images that can be used in association with an embodiment of the present invention is provided. The information processing environment can include one or more of an imaging apparatus 102, one or more servers (e.g., an image server 105 and a report server 107), and computing devices (e.g., a medical care workstation (WS) 104 and an interpretation WS 103) communicatively coupled together via a network 109. It should be understood that two or more of the aforementioned components may be combined in a single unit. Network 109 can be made up of telecommunication network technologies that are based on physically wired, optical, and/or wireless radio-frequency methods that may be arranged in a variety of network topologies. In general, network 109 can be any set of digital interconnections that allow computing devices to use common communication protocols to communicate with each other.

Imaging apparatus 102 may be one or more imaging devices that include an emitter 130 and a sensor 120. Imaging apparatus 102 is configured to scan the body of a subject by exposing the subject to signals emitted by emitter 130, capturing those signals (or signal reflections) via sensor 120, and thereby obtain detailed internal images of the subject's body (e.g., images of the anatomy and the physiological processes inside the body). The medical images generated by imaging apparatus 102 may be stored in imaging database 106 and/or other database that is communicatively coupled to network 109. For example, imaging apparatus 102 is configured to generate sequential tomographic images of a subject. Imaging apparatus 102 may capture tomographic images along one or more anatomical planes (e.g., the transaxial plane, coronal plane, sagittal plane, median plane, parasagittal plane, and other anatomical planes) of subjects.

Applicable signals emitted by emitter 130 include, but are not limited to, strong magnetic fields, magnetic field gradients, radio waves, X-rays, and ionizing radiation. Imaging apparatus 102 preferably generates heterogeneous sequential tomographic images of the subject. Imaging apparatus 102 can generate medical images using one or more medical imaging techniques that include, but are not limited to, X-ray, CT scan, MRI, PET, MIP, and/or other medical imaging technique capable of generating internal images of a subject's body or part thereof.

The servers may each include a software program providing a database management system (DBMS) installed therein. In some embodiments, image server 105 and report server 107 can be included in one or more computing devices. The servers may include one or more storage media (e.g., flash memory, solid-state drive (SSD), or hard disk drive (HDD)) for storing information. For example, image server 105 can include an image database (DB) 106 for at least storing medical images. Report server 107 may include a report DB 108 for at least storing processed reports. Medical care WS 104 can be used by healthcare professionals (e.g., doctors and nurses) to at least observe medical images in detail, view interpretation reports, and/or generate electronic medical records.

Medical care WS 104 may transmit a viewing request for medical images to image server 105, display the received medical images, transmit a viewing request for the associated interpretation report(s) to report server 107, and/or display the received interpretation report. Medical care WS 104 may perform the above processes via executing software programs for respective processes. The steps described in the instant disclosure may be performed by one or more control circuits and/or processors. The interpretation reports can include images analyzed by an information processing program 110 included in interpretation WS 103 (or other computing device communicatively coupled to network 109).

Interpretation WS 103 is a computing device that can be configured to analyze or interpret one or more medical images (e.g., stored in image DB 106 or other data store communicatively coupled to network 109) as described in the present disclosure. Interpretation WS 103 may be used by user (e.g., a healthcare professional such as a radiologist) to interpret one or more medical images and/or create interpretation reports. In certain instances, interpretation WS 103 may be proximate to the other elements in information processing environment 100, such as in a medical center or hospital. In other instances, interpretation WS 103 may be remote to the other elements in information processing environment 100. For example, interpretation WS 103 may be in a home or remote office of a medical professional (e.g., a radiologist). Interpretation WS 103, or other processor(s) communicatively coupled to network 109, can perform steps defined by an information processing program 110. As will be discussed in more detail below, information processing program 110 is computer code that enables the display, navigation, examination, and/or annotation of sets of medical images. Information processing program 110 can be stored in a database communicatively coupled to the interpretation WS 103. Alternatively, information processing program 110 can be stored in one or more databases that are communicatively coupled to network 109.

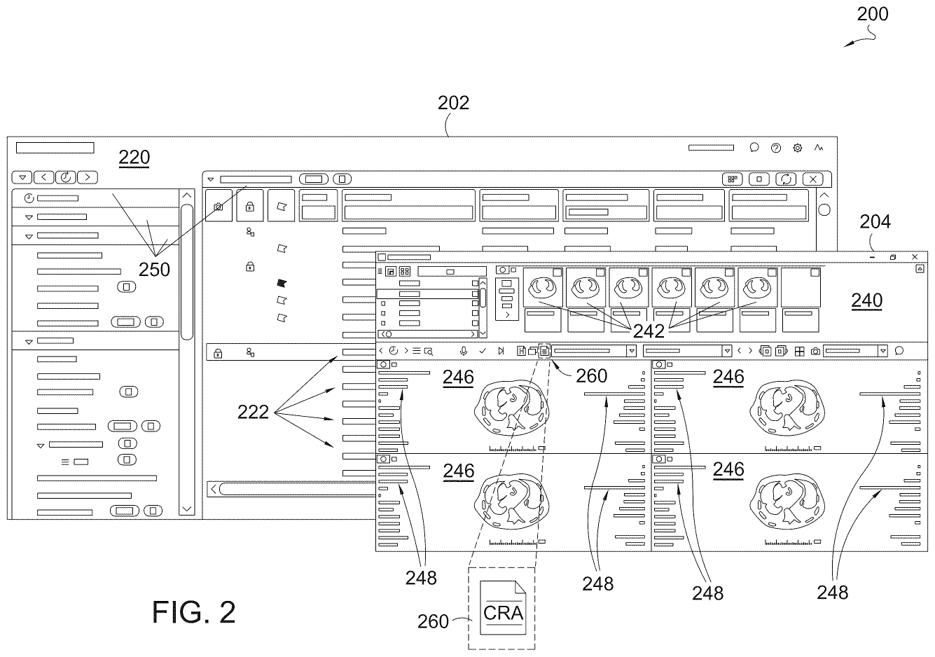

Information processing program 110 may be or may include a picture archiving and communication system (PACS) 200. Referring now to FIGS. 2-8 and 8A-8D, embodiments of the present invention provide computer-implemented methods, systems, and computer program products for automated annotation of vessel diagrams within PACS 200. PACS 200 includes a plurality of imaging studies including a plurality of medical image files, each medical image file including a medical image 242 captured by one or more medical imaging apparatus 102 and stored in one or more databases 106, 108 that are communicatively coupled to network 109. Each of the plurality of medical image files includes a medical image 242 and corresponding metadata 248 (e.g., digital imaging and communications in medicine (DICOM) formatted data). The metadata may comprise a plurality of data objects corresponding to information entities (IEs). For example, the data objects may correspond to information entities such as a patient, a study, a medical device, a physician's visit, a series of images, a patent schedule, a document, a diagnostic interpretation, etc. The data objects may include attributes consisting of a tag, a value representation describing the data type and format, and an alphanumeric data value.

After logging in to PACS 200 on interpretation WS 103 or medical care WS 104, a worklist graphical user interface (GUI) 220 may be provided in a first window 202 displayed on a display of WS 103, 104. Worklist GUI 220 may include one or more panes 250 displaying one or more lists of a plurality of imaging studies 222. Each of plurality of imaging studies 222 may be associated with one or more medical image files, each medical image file including a medical image 242 of one or more anatomical structures of a subject (e.g., person) captured by one or more medical imaging apparatuses 102.

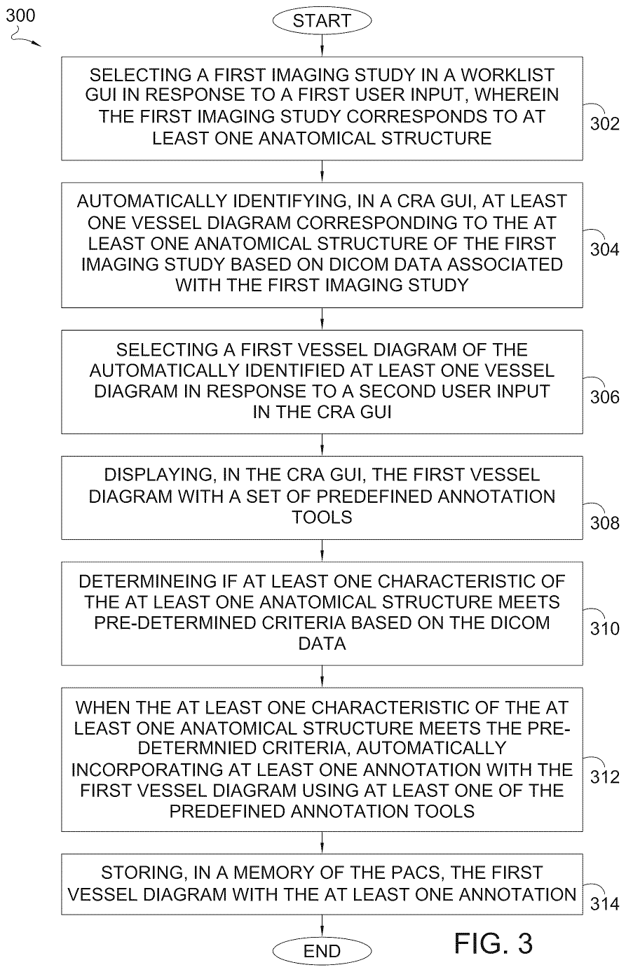

With additional reference to a computer-implemented method 300 shown in FIG. 3, when a first imaging study 222 is selected from worklist GUI 220, at operational step 302, a second window 204 providing a viewer GUI 240 may be displayed on the display of WS 103, 104. Viewer GUI 240 includes at least one pane 246 that is configured for displaying at least one medical image 242 of the selected at least one imaging study 222. The at least one medical image 242 is displayed in at least one pane 246 for viewing by medical professionals for the purpose of reviewing, evaluating and diagnosing any medical conditions that may be present in the images subject. The at least one medical image 242 includes a metadata overlay layer comprising metadata 248. In some embodiments, viewer GUI 240 may include a plurality of panes 246, wherein each pane displays an image of the plurality of medical images 242 of selected imaging study 222.

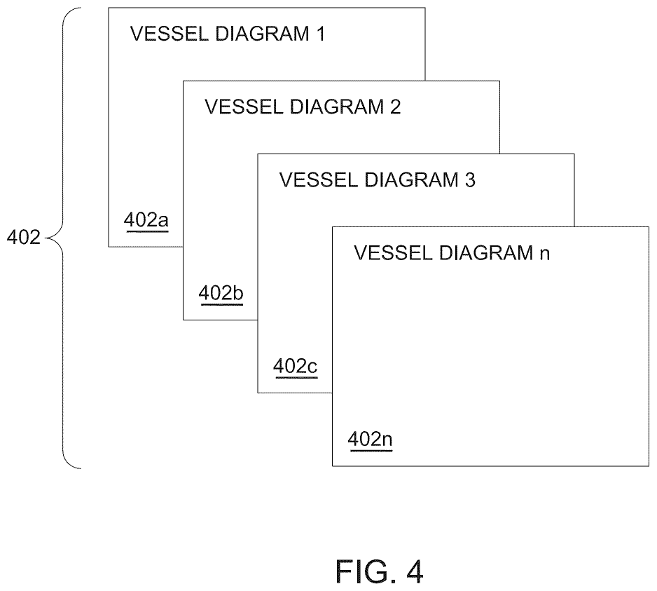

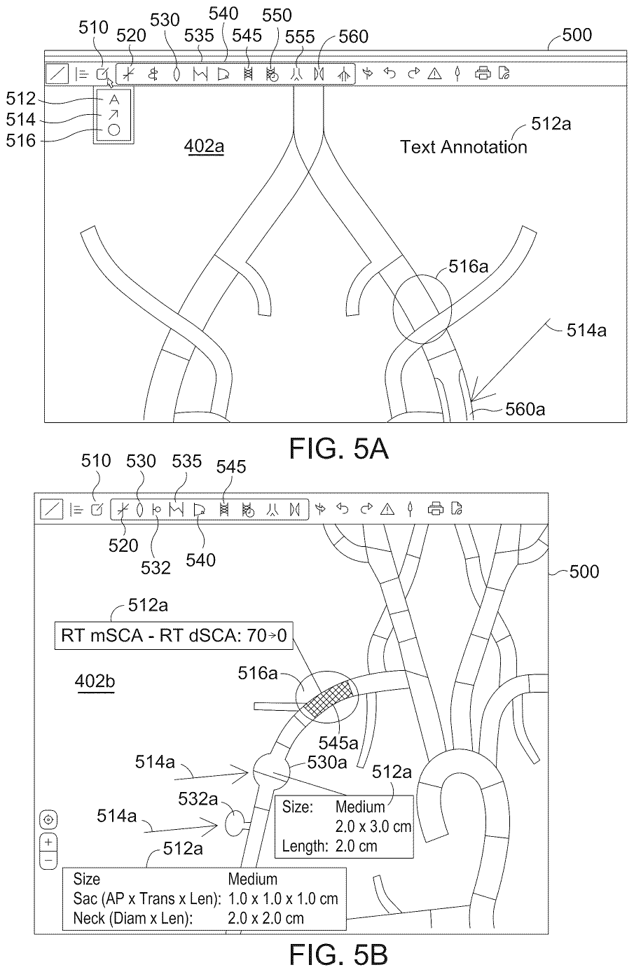

Viewer GUI 240 also includes a clinical reporting application (CRA) icon 260. Selection of CRA icon 260, in response to a user input, launches a CRA GUI 500 (e.g., FIG. 5A) and automatically identifies one or more vessel diagrams 402 (FIG. 4) corresponding to at least one anatomical structure found within medical images 242 of the first imaging study 222 based on DICOM data, at operational step 304. The DICOM data comprises a plurality of DICOM data objects corresponding to information entities (IEs) associated with the first imaging study. The DICOM data may include patient IEs, study IEs, series IEs, equipment IEs, frame of reference IEs, image IEs, overlay IEs, modality IEs, presentation state IEs, waveform IEs, structured reporting (SR) IEs, raw data IEs, encapsulated document IEs, real world value mapping IEs, surface IEs, measurement IEs, and the like. SR IEs are a part of the DICOM standard that allows for the creation, storage, and communication of structured medical reports. Unlike conventional text-based reports, SR IEs provide a standardized, structured format that enhances the consistency and usability of report information. Accordingly, DICOM data may include pathologic information, diagnostic information, or therapeutic information.

Embodiments of the present invention provide computer-implemented methods, systems, and computer program products for automated annotation of vessel diagrams within PACS 200. PACS 200 may include a plurality of imaging studies 222 including at least one medical image captured by a medical imaging apparatus, wherein the at least one medical image includes associated DICOM data. Each imaging study 222 may include one or more series of medical image files. Each series of medical image files may include a plurality of medical image files. Each medical image file includes a medical image 242 captured by a medical imaging apparatus (e.g., imaging apparatus 102) and associated metadata (e.g., DICOM formatted data).

At operational step 306, and with additional reference to FIGS. 4 and 5A, a first vessel diagram 402a of the automatically identified one or more vessel diagrams 402 is selected in response to a user input in CRA GUI 500. It should be understood that the one or more vessel diagrams 402 may include an arbitrary number of vessel diagrams (e.g., first vessel diagram 402a, second vessel diagram 402b, third vessel diagram 402c, to nth vessel diagram 402n). For example, a user may select a vessel diagram identifier, such as a textual description, from a list of vessel diagram identifiers representing the automatically identified one or more vessel diagrams 402, or a user may select a graphical representation of a vessel diagram (e.g., a first vessel diagram 402a or an arbitrary second vessel diagram 402n) of graphical representations corresponding to automatically identified one or more vessel diagrams 402.

At operational step 308, first vessel diagram 402a is displayed in CRA GUI 500 along with a set of predefined annotation tools. In certain embodiments, the set of predefined annotation tools may include annotation tools 510 (e.g., a text tool 512, an arrow tool 514, and an ellipse tool 516), a vessel tool 520, a graft tool 535, a collateral tool 540, medical device tools (e.g. a stent tool 545, a previous stent tool 550, a stent graft tool 555, or the like), and lesion tools (e.g., an aneurysm tool 530, a stenosis tool 560, or the like). In some embodiments, aneurysm tool 530 may include a saccular aneurysm tool 532, a fusiform aneurysm tool 530, a dissection aneurysm (not shown), or the like. In other embodiments, the aneurysm tool 530 may allow the user to indicate a type of aneurysm when incorporating aneurysm FIG. 530a or 532a into first vessel diagram 402a.

At operational step 310, it is determined whether at least one characteristic of the at least one anatomical structure of selected imaging study 222 meets a pre-determined criteria based on the DICOM data (e.g., based on the measurement IEs, the SR IEs, or the like). For example, the at least one characteristic of the at least one anatomical structure of selected imaging study 222 may be a maximum diameter or a minimum diameter of a vessel in the at least one anatomical structure. In other instances, the at least one characteristic of the at least one anatomical structure of selected imaging study 222 may also be the presence of a medical device, such as a stent, a valve, electrical leads, implantable sensors, or the like.

When at least one anatomical structure of selected imaging study 222 meets the pre-determined criteria based on the DICOM data, at least one annotation may be automatically incorporated with the first vessel diagram using at least one of the predefined annotation tools, at operational step 312. The at least one annotation includes metadata indicating coordinates of the at least one annotation on first vessel diagram 402a, a type of the at least one annotation, or a content of the at least one annotation. The coordinates of the at least one annotation on first vessel diagram 402a enable to annotation to remain in place within the first vessel diagram when the first is saved, reopened, or incorporated into a clinical report. The type of annotation may include a text annotation, an arrow annotation, and an ellipse annotation, a vessel annotation, a graft annotation, a collateral annotation, medical device annotations (e.g. a stent annotation, a previous stent annotation, a stent graft annotation, or the like), and lesion annotation (e.g., an aneurysm annotation, a stenosis annotation, or the like).

Further, after the at least one annotation is incorporated with first vessel diagram 402a, first vessel diagram 402a is stored, at operational step 314, with the at least one annotation, including the coordinates of the at least one annotation on the first vessel diagram, the type of the at least one annotation, or the content of the at least one annotation, in a memory of the PACS (e.g., in image DB 106, report DB 108, system memory 836, or the like).

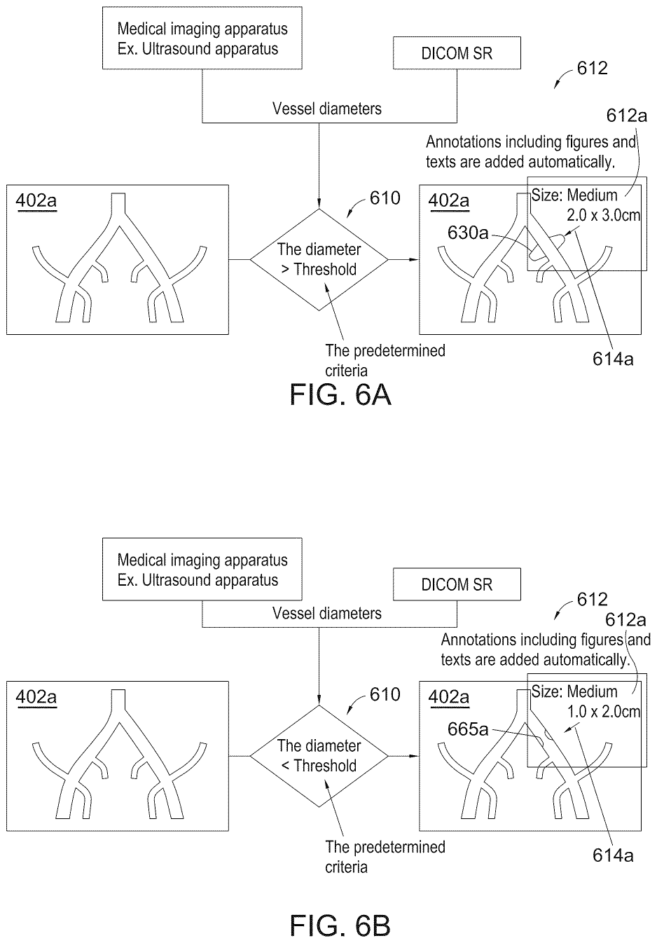

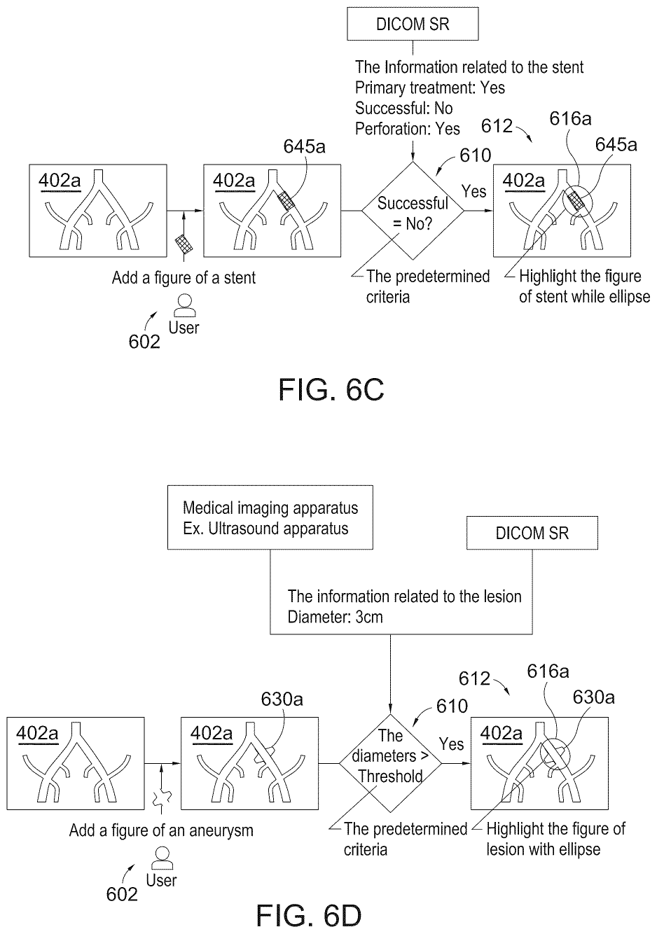

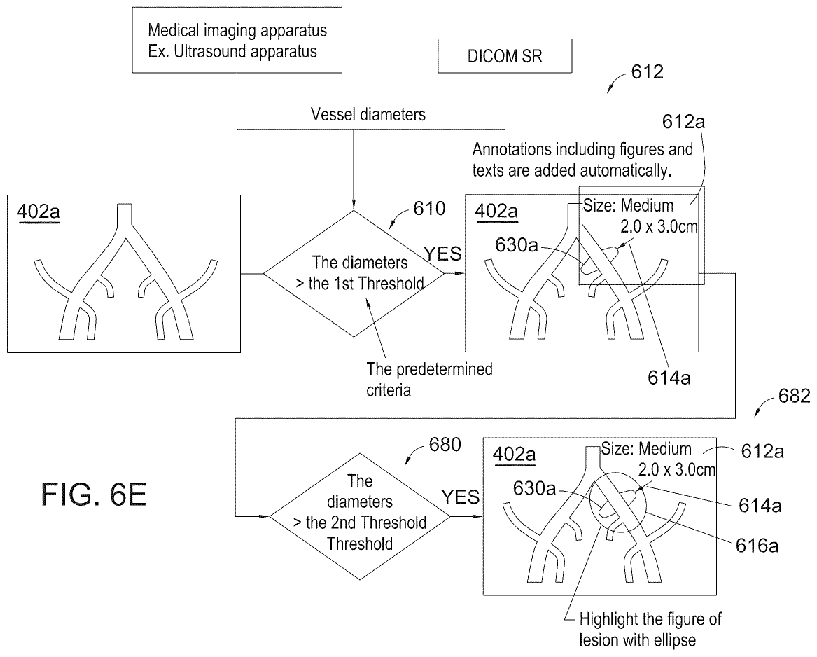

For example, in some instances, the at least one characteristic of the at least one anatomical structure of selected imaging study 222 may be a maximum diameter and the pre-determined criteria may be greater than a threshold value. For example, with reference to FIG. 6A, when an aneurysm is present along a vessel, the maximum diameter of the vessel at the aneurysm may be greater than a nominal diameter of the vessel. Accordingly, the maximum diameter of the vessel as identified in a DICOM SR IE may be compared to a threshold value, at 610. If the maximum diameter of the vessel is greater than the threshold value, at least one annotation may be automatically incorporated with first vessel diagram 402a using at least one of the predefined annotation tools, at 612. That is, an annotation text 612a, an annotation arrow 614a, an annotation aneurysm FIG. 630a, or the like may be automatically incorporated with first vessel diagram 402a when the maximum diameter of the vessel is greater than the threshold value.

In other instances, the at least one characteristic of the at least one anatomical structure of selected imaging study 222 may be a minimum diameter and the pre-determined criteria may be less than or equal to a threshold value. For example, with reference to FIG. 6B, when a stenosis is present along a vessel, the minimum diameter of the vessel at the stenosis may be less than or equal to a nominal diameter of the vessel. Accordingly, the minimum diameter of the vessel as identified in a DICOM SR IE may be compared to a threshold value, at 610. If the minimum diameter of the vessel is less than or equal to the threshold value, at least one annotation may be automatically incorporated with first vessel diagram 402a using at least one of the predefined annotation tools, at 612. That is, an annotation text 612a, an annotation arrow 614a, an annotation stenosis FIG. 630a, or the like may be automatically incorporated with first vessel diagram 402a when the minimum diameter of the vessel is less than or equal to the threshold value.

In yet other instances, the at least one characteristic of the at least one anatomical structure of selected imaging study 222 may be a presence of a medical device (e.g., a stent, a valve, electrical leads, implantable sensors, or the like) and the pre-determined criteria may be whether a state of the device is non-functional. For example, with reference to FIG. 6C, when a stent is present along a vessel, the stent may be functional and successful or non-functional due to a perforation. Accordingly, the functionality of the stent, as identified in a DICOM SR IE, may be compared to the pre-determined criteria, at 610. If a stent is present and non-functional, at least one annotation may be automatically incorporated with first vessel diagram 402a using at least one of the predefined annotation tools, at 612. That is, an annotation ellipse 616a or the like may be automatically incorporated with first vessel diagram 402a when the minimum diameter of the vessel is less than or equal to the threshold value.

As discussed above, the addition of annotations to first vessel diagram 402a may be automated. However, annotations may also be added manually. For example, at 602, a user (e.g., a radiologist or other health care provider) may manually add a stent FIG. 545a (FIGS. 5B), 645a (FIG. 6C), using a stent tool 545, to a segment of the anatomical structure in first vessel diagram 402a.

Similarly, with reference to FIG. 6D, a user (e.g., a radiologist or other health care provider) may manually add an aneurysm FIGS. 530a, 630a, using an aneurysm tool 530, to a segment of the anatomical structure in first vessel diagram 402a. The maximum diameter of the vessel as identified in a DICOM SR IE may be compared to a threshold value, at 610. If the maximum diameter of the vessel is greater than the threshold value, at least one annotation may be automatically incorporated with first vessel diagram 402a using at least one of the predefined annotation tools, at 612. That is, an annotation ellipse 616a or the like may be automatically incorporated with first vessel diagram 402a when the maximum diameter of the vessel is greater than the threshold value. Accordingly, within first vessel diagram 402a some annotations (e.g., aneurysm FIG. 630a) may be manually added, while the addition of some annotations (e.g., annotation ellipse 616a) may be automated. When annotation text 512a, 612a is automatically added to first vessel diagram 402a, a content of annotation text 512a, 612a (e.g., characteristics of a vascular lesion or medical device) may originate in the DICOM data (e.g., the SR IE, the measurement IE, or the like).

In certain embodiments, the at least one characteristic of the at least one anatomical structure of selected imaging study 222 may be compared with more than one pre-determined criteria based on the DICOM data. For example, with reference to FIG. 6E, a diameter of the vessel as identified in a DICOM SR IE may be compared to a first threshold value, at 610. When the diameter of the vessel meets the first predetermined criteria (e.g., the maximum value of the vessel is greater than the first threshold value), a first annotation may be automatically incorporated with first vessel diagram 402a using at least one of the predefined annotation tools, at 612. That is, an annotation text 612a, an annotation arrow 614a, an annotation aneurysm FIG. 630a, or the like may be automatically incorporated with first vessel diagram 402a when the maximum diameter of the vessel is greater than the first threshold value.

Additionally, the diameter of the vessel as identified in a DICOM SR IE may be compared to a second threshold value, at 680. When the diameter of the vessel meets the second predetermined criteria (e.g., the maximum value of the vessel is greater than the second threshold value), a second annotation may be automatically incorporated with first vessel diagram 402a using at least one of the predefined annotation tools, at 682. That is, an annotation ellipse 616a or the like may also be automatically incorporated with first vessel diagram 402a when the maximum diameter of the vessel is greater than the second threshold value.

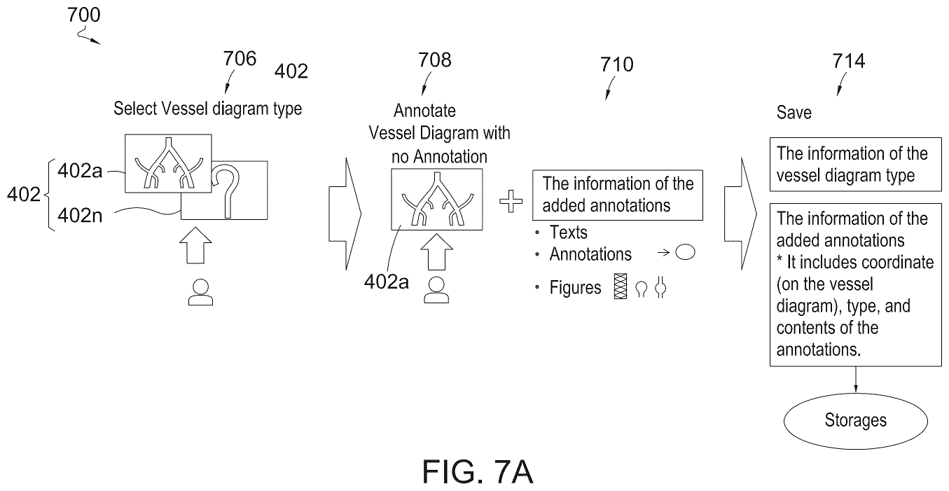

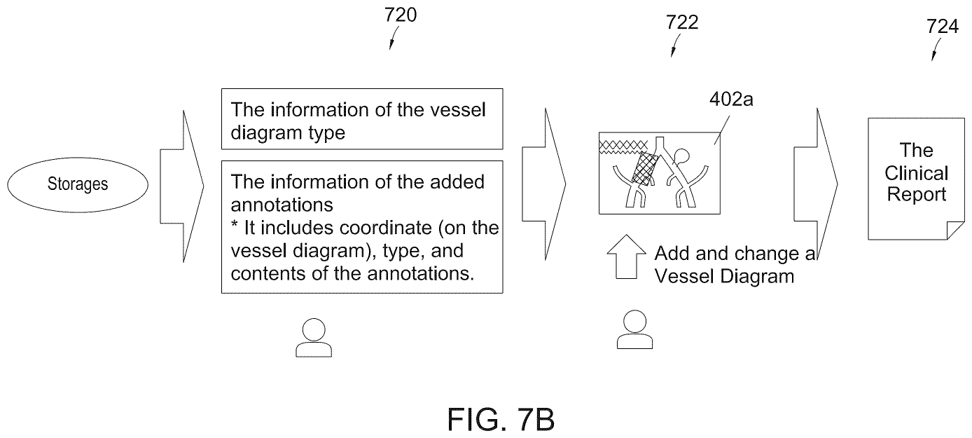

FIGS. 7A-7B are alternative depictions illustrating operational steps for automated annotation of vessel diagrams within a PACS. With additional reference to a method 700 shown in FIGS. 7A-7B, selection of CRA icon 260 (FIG. 2) launches CRA GUI 500 (e.g., 5A, 5B) and automatically identifies one or more vessel diagrams 402 (FIG. 4) corresponding to at least one anatomical structure found within medical images 242 of the first imaging study 222 based on DICOM data comprises a plurality of DICOM data objects corresponding to information entities (IEs) associated with the first imaging study. As previously mentioned, the one or more vessel diagrams 402 may include a plurality of vessel diagrams (e.g. first vessel diagram 402a, second vessel diagram 402b, and additional vessel diagrams 402n). First vessel diagram 402a of the automatically identified one or more vessel diagrams 402 is selected in response to a user input in CRA GUI 500, at operational step 706.

At operational step 708, first vessel diagram 402a is displayed in CRA GUI 500 along with a set of predefined annotation tools. As previously mentioned, with reference to FIGS. 5A and 5B, the set of predefined annotation tools may include annotation tools 510 (e.g., text tool 512, arrow tool 514, and ellipse tool 516), vessel tool 520, graft tool 535, collateral tool 540, medical device tools (e.g. stent tool 545, previous stent tool 550, stent graft tool 555, or the like), and lesion tools (e.g., aneurysm tool 530, stenosis tool 560, or the like). In some embodiments, aneurysm tool 530 may include saccular aneurysm tool 532, fusiform aneurysm tool 530, dissection aneurysm (not shown), or the like. In other embodiments, aneurysm tool 530 may allow the user to indicate a type of aneurysm when incorporating aneurysm FIG. 530a or 532a into first vessel diagram 402a.

As described above, annotations may be made to first vessel diagram 402a. The addition of annotations to first vessel diagram 402a may be automated and may be added manually. The at least one annotation includes metadata indicating coordinates of the at least one annotation on the first vessel diagram, a type of the at least one annotation, or a content of the at least one annotation. The type of annotation may include a text annotation, an arrow annotation, and an ellipse annotation, a vessel annotation, a graft annotation, a collateral annotation, medical device annotations (e.g. a stent annotation, a previous stent annotation, a stent graft annotation, or the like), and lesion annotation (e.g., an aneurysm annotation, a stenosis annotation, or the like).

After the at least one annotation is incorporated with first vessel diagram 402a, first vessel diagram 402a is stored, at operational step 714, with the at least one annotation, including the coordinates of the at least one annotation on the first vessel diagram, the type of the at least one annotation, or the content of the at least one annotation, in a memory of the PACS (e.g., in image DB 106, report DB 108, system memory 836, or the like).

At operational step 720, first vessel diagram 402a, with the at least one annotation including the metadata indicating the coordinates of the at least one annotation on the first vessel diagram, the type of the at least one annotation, or the content of the at least one annotation is retrieved from the memory of the PACS. First vessel diagram 402s with the at least one annotation is displayed in the CRA GUI with the set of predefined annotation tools.

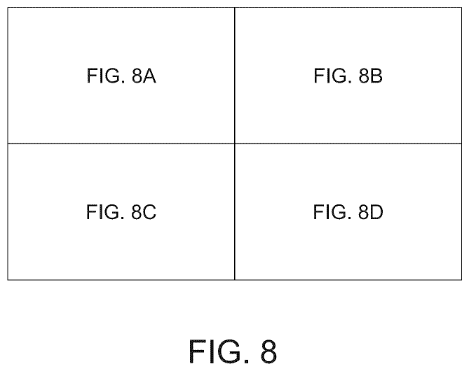

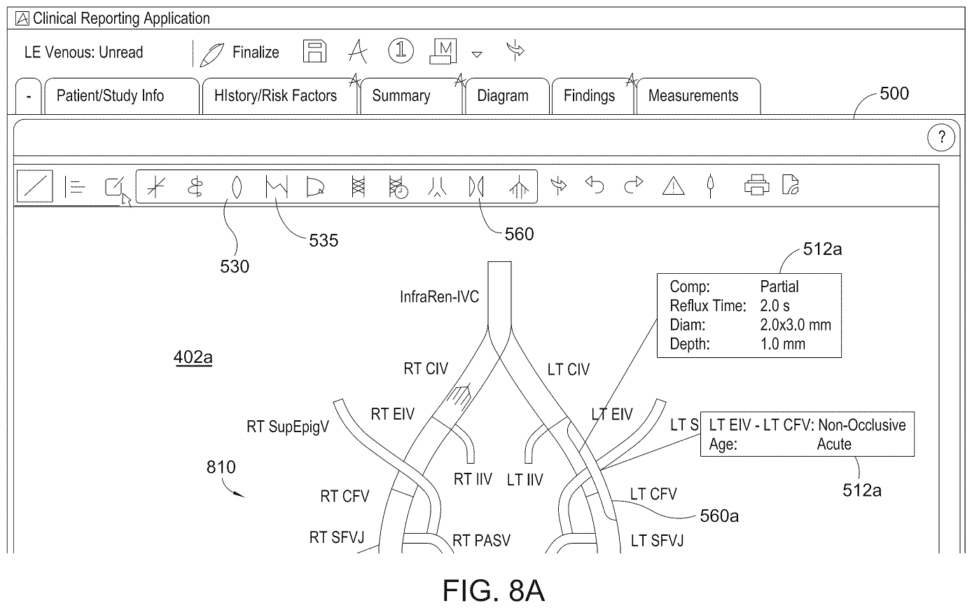

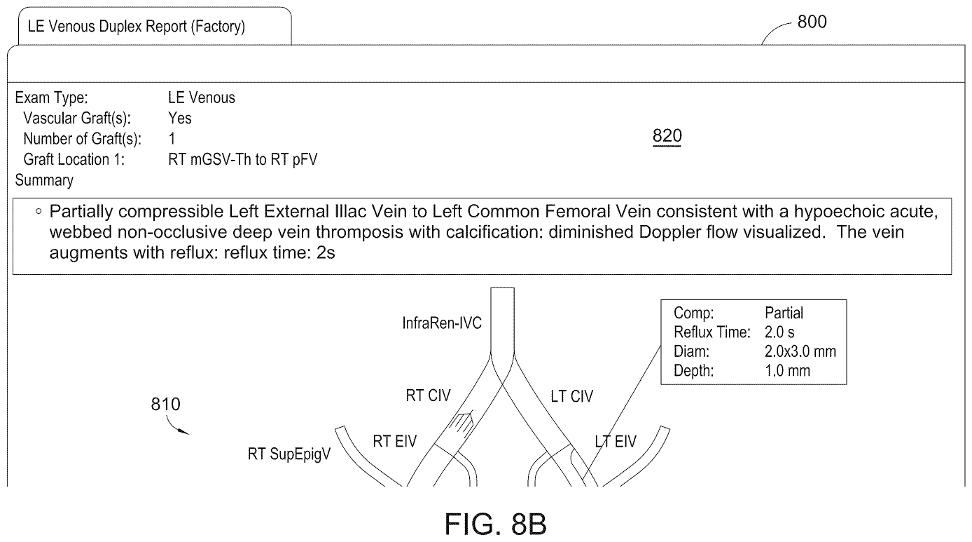

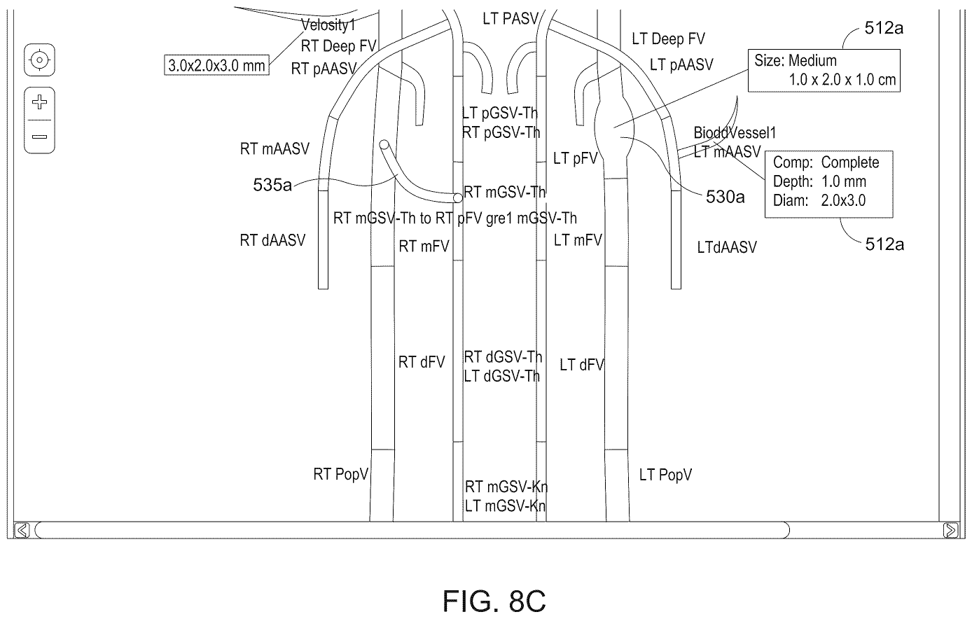

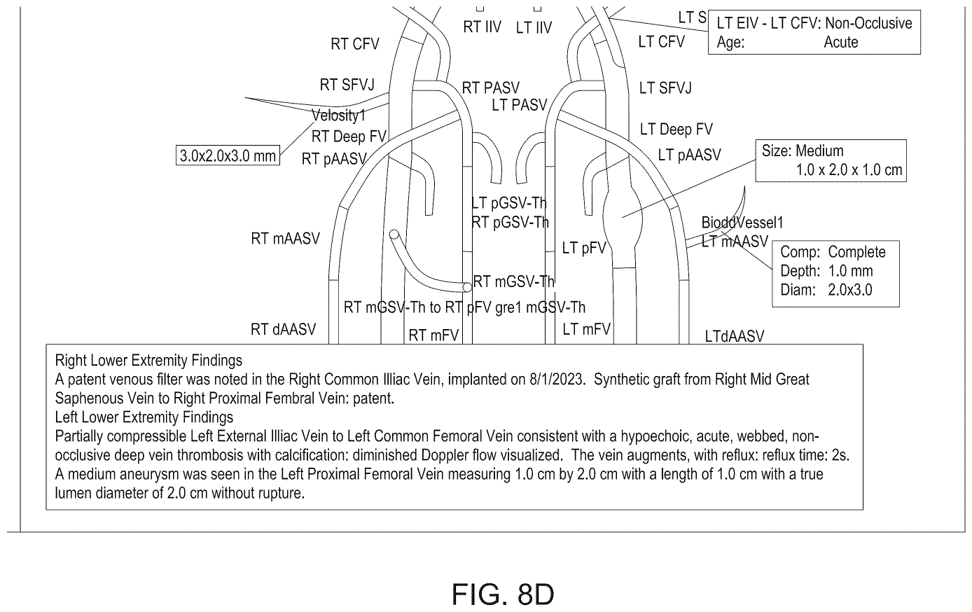

At operational step 722, with additional reference to FIGS. 8 and 8A-8D, a user input is received modifying the at least one annotation of first vessel diagram 402a, and first vessel diagram 402a with the modified at least one annotation 810 is stored. Modifying of the at least one annotation of the first vessel diagram comprises adding one or more additional annotations, changing at least one of coordinates of the at least one annotation on the first vessel diagram, changing a type of the at least one annotation, or changing a content of the at least one annotation.

For example, first vessel diagram 402a with the modified at least one annotation 810 may include the addition or modification of annotation text 512a, aneurysm FIG. 530a via aneurysm tool 530, graft FIG. 535a via graft tool 535, stenosis FIG. 560a via stenosis tool 560, segment names, and anatomical landmarks.

At operational step 724, a clinical report 820 corresponding to the at least one anatomical structure of the first imaging study is generated in a report window 800 based on the DICOM data associated with the first imaging study, and first vessel diagram 402a with the modified at least one annotation 810 is incorporated in to the generated clinical report 820. Clinical report 820 may incorporate first vessel diagram 402a with the modified at least one annotation 810, as well as any number of other annotated diagrams.

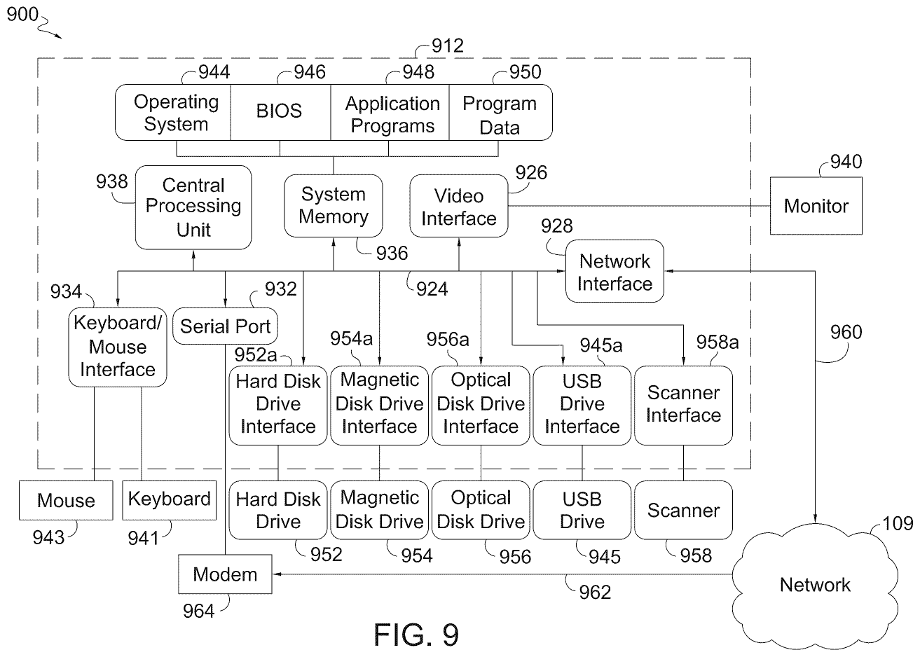

Referring to FIG. 9, an exemplary computing environment 900 is shown that can be utilized through programming to implement any of the processing thus far described. The computing environment 900 may comprise a computer 912 including a system bus 924 that couples a video interface 926, network interface 928, one or more serial ports 932, a keyboard/mouse interface 934, and a system memory 936 to a Central Processing Unit (CPU) 938. Computer 912 may also include a Graphics Processing Unit (GPU) or one or more other special or general purpose processing units. A monitor or display 940 is connected to bus 924 by video interface 926 and provides the user with a graphical user interface to view, edit, and otherwise manipulate items displayed on computer 912. The graphical user interface allows the user to enter commands and information into computer 912 using a keyboard 941 and a user interface selection device 943, such as a mouse or other pointing device. Keyboard 941 and user interface selection device are connected to bus 924 through keyboard/mouse interface 934. The display 940 and user interface selection device 943 are used in combination to form the graphical user interface, which may allow a user to implement at least a portion of the present invention. Other peripheral devices may be connected to computer 912 through serial port 932 or universal serial bus (USB) drives 945 to transfer information to and from computer 912.

The system memory 936 is also connected to bus 924 and may include ROM, RAM, an operating system 944, a basic input/output system (BIOS) 946, application programs 948 and program data 950. The computer 912 may further include a hard disk drive 952 for reading from and writing to a hard disk, a magnetic disk drive 954 for reading from and writing to a removable magnetic disk (e.g., floppy disk), and an optical disk drive 956 for reading from and writing to a removable optical disk (e.g., CD ROM or other optical media). The computer 912 may also include USB drives 945 and other types of drives for reading from and writing to flash memory devices (e.g., compact flash, memory stick/PRO and DUO, SD card, multimedia card, smart media card), and a scanner 958 for scanning items such as digital images to be downloaded to computer 912. A hard disk interface 952a, magnetic disk drive interface 954a, an optical drive interface 956a, a USB drive interface 945a, and a scanner interface 958a operate to connect bus 924 to hard disk drive 952, magnetic disk drive 954, optical disk drive 956, USB drive 945 and a scanner 958, respectively. Each of these drive components and their associated computer-readable media may provide computer 912 with non-volatile storage of computer-readable instruction, program modules, data structures, application programs, an operating system, and other data for the computer 912. In addition, it will be understood that computer 912 may also utilize other types of computer-readable media in addition to those types set forth herein, such as digital video disks, random access memory, read only memory, other types of flash memory cards, magnetic cassettes, and the like.

Network interface 928 provides a communication path 960 between bus 924 and network 109, which allows notifications, information and other data to be communicated through network 109 from any of the previously identified devices, and optionally saved in a memory, to the computer 912. This type of logical network connection is commonly used in conjunction with a local area network. Images may also be communicated from bus 924 through a communication path 962 to network 109 using serial port 932 and a modem 964. Using a modem connection between the computer 912 and other computing devices, databases, or the like may be used in conjunction with a wide area network or the Internet. It will be appreciated that the network connections shown herein are merely exemplary, and it is within the scope of the present invention to use other types of network connections between computer 912 and other computing devices including both wired and wireless connections.

As discussed above, embodiments of the present invention provide improved methods and systems within PACS that enable creation of detailed reports that include automated annotation of vessel diagrams and precise information on lesions and measurements of blood vessels.

Based on the foregoing, method, computer system, and program product have been disclosed in accordance with the present invention. However, numerous modifications and substitutions can be made without deviating from the scope of the present invention. Therefore, the present invention has been disclosed by way of example and not limitation.

What is claimed is:

1. A computer-implemented method for automated annotation of vessel diagrams within a picture archiving and communication system (PACS), the PACS comprising at least one imaging study including at least one medical image captured by a medical imaging apparatus, wherein the at least one medical image includes associated digital imaging and communications in medicine (DICOM) data, the computer-implemented method comprising:
selecting a first imaging study in a worklist graphical user interface (GUI) in response to a first user input, wherein the first imaging study corresponds to at least one anatomical structure;
automatically identifying, in a clinical reporting application (CRA) GUI, at least one vessel diagram corresponding to the at least one anatomical structure of the first imaging study based on the DICOM data associated with the first imaging study;
selecting a first vessel diagram of the automatically identified at least one vessel diagram in response to a second user input in the CRA GUI ;
displaying, in the reporting CRA GUI, the first vessel diagram with a set of predefined annotation tools;
determining if at least one characteristic of the at least one anatomical structure meets pre-determined criteria based on the DICOM data;
when the at least one characteristic of the at least one anatomical structure meets the pre-determined criteria, automatically incorporating at least one annotation with the first vessel diagram using at least one of the predefined annotation tools; and
storing, in a memory of the PACS, the first vessel diagram with the at least one annotation.

2. The computer-implemented method of claim 1, wherein the set of predefined annotation tools includes at least one of annotation tools, a vessel tool, an aneurysm tool, a graft tool, a collateral tool, a stent tool, a previous stent tool, a stent graft tool, or a lesion or stenosis tool.

3. The computer-implemented method of claim 2, wherein the annotation tools include at least one of an ellipse tool, an arrow tool, or a text tool.

4. The computer-implemented method of claim 1, wherein the at least one annotation includes metadata comprising at least one of coordinates of the at least one annotation on the first vessel diagram, a type of the at least one annotation, or a content of the at least one annotation.

5. The computer-implemented method of claim 1, the computer-implemented method further comprising:
retrieving, from the memory of the PACS, the first vessel diagram with the at least one annotation;
displaying, in the CRA GUI, the first vessel diagram with the at least one annotation and the set of predefined annotation tools;
receiving a user input modifying the at least one annotation of the first vessel diagram; and
storing the first vessel diagram with the modified at least one annotation.

6. The computer-implemented method of claim 5, wherein the modifying of the at least one annotation of the first vessel diagram comprises: adding one or more additional annotations, changing at least one of coordinates of the at least one annotation on the first vessel diagram, a type of the at least one annotation, or a content of the at least one annotation.

7. The computer-implemented method of claim 5, the computer-implemented method further comprising:
generating a clinical report corresponding to the at least one anatomical structure of the first imaging study based on the DICOM data associated with the first imaging study; and
incorporating the first vessel diagram with the modified at least one annotation in the generated clinical report.

8. The computer-implemented method of claim 1, wherein the DICOM data includes pathologic information, diagnostic information, or therapeutic information.

9. The computer-implemented method of claim 1, wherein the at least one characteristic of the at least one anatomical structure is a maximum diameter of a vessel, and
wherein the predetermined criteria is greater than a threshold value.

10. The computer-implemented method of claim 1, wherein the at least one characteristic of the at least one anatomical structure is a minimum diameter of a vessel, and
wherein the predetermined criteria is less than or equal to a threshold value.

11. The computer-implemented method of claim 1, wherein the at least one characteristic of the at least one anatomical structure is a presence of a medical device, and
wherein the predetermined criteria is the medical device is non-functional.

12. A computer system for automated annotation of vessel diagrams within a picture archiving and communication system (PACS), the PACS comprising at least one imaging study including at least one medical image captured by a medical imaging apparatus, wherein the at least one medical image includes associated digital imaging and communications in medicine (DICOM) data, the computer system comprising:
one or more computer processors;
one or more non-transient, computer-readable storage media;
program instructions stored on the one or more non-transient, computer-readable storage media for execution by at least one of the one or more computer processors, the program instructions comprising:
program instructions to:
select a first imaging study in a worklist graphical user interface (GUI) in response to a first user input, wherein the first imaging study corresponds to at least one anatomical structure;
automatically identify, in a clinical reporting application (CRA) GUI, at least one vessel diagram corresponding to the at least one anatomical structure of the first imaging study based on the DICOM data associated with the first imaging study;
select a first vessel diagram of the automatically identified at least one vessel diagram in response to a second user input in the CRA GUI;
display, in the CRA GUI, the first vessel diagram with a set of predefined annotation tools;
determine if at least one characteristic of the at least one anatomical structure meets pre-determined criteria based on the DICOM data;
when the at least one characteristic of the at least one anatomical structure meets the pre-determined criteria, automatically incorporate at least one annotation with the first vessel diagram using at least one of the predefined annotation tools; and
store, in a memory of the PACS, the first vessel diagram with the at least one annotation.

13. The computer system of claim 12, wherein the set of predefined annotation tools includes at least one of annotation tools, a vessel tool, an aneurysm tool, a graft tool, a collateral tool, a stent tool, a previous stent tool, a stent graft tool, or a lesion or stenosis tool.

14. The computer system of claim 13, wherein the annotation tools include at least one of an ellipse tool, an arrow tool, or a text tool.

15. The computer system of claim 12, wherein the at least one annotation includes metadata comprising at least one of coordinates of the at least one annotation on the first vessel diagram, a type of the at least one annotation, or a content of the at least one annotation.

16. The computer system of claim 12, wherein the program instructions further comprise program instructions to:
retrieve, from the memory of the PACS, the first vessel diagram with the at least one annotation;
display, in the CRA GUI, the first vessel diagram with the at least one annotation and the set of predefined annotation tools;
receive a user input modifying the at least one annotation of the first vessel diagram; and
store the first vessel diagram with the modified at least one annotation.

17. The computer system of claim 16, wherein the modifying of the at least one annotation of the first vessel diagram comprises: adding one or more additional annotations, changing at least one of coordinates of the at least one annotation on the first vessel diagram, a type of the at least one annotation, or a content of the at least one annotation.

18. The computer system of claim 16, wherein the program instructions further comprise program instructions to:
generate a clinical report corresponding to the at least one anatomical structure of the first imaging study based on the DICOM data associated with the first imaging study; and
incorporate the first vessel diagram with the modified at least one annotation in the generated clinical report.

19. The computer system of claim 12, wherein the DICOM data includes pathologic information, diagnostic information, or therapeutic information.

20. The computer system of claim 12, wherein the at least one characteristic of the at least one anatomical structure is a maximum diameter of a vessel, and
wherein the predetermined criteria is greater than a threshold value.

21. The computer system of claim 12, wherein the at least one characteristic of the at least one anatomical structure is a minimum diameter of a vessel, and
wherein the predetermined criteria is less than or equal to a threshold value.

22. The computer system of claim 12, wherein the at least one characteristic of the at least one anatomical structure is a presence of a medical device, and
wherein the predetermined criteria is the medical device is non-functional.

23. A computer program product for automated annotation of vessel diagrams within a picture archiving and communication system (PACS), the PACS comprising at least one imaging study including the at least one medical image captured by a medical imaging apparatus, wherein the at least one medical image includes associated digital imaging and communications in medicine (DICOM) data, the computer program product comprising a non-transient computer readable storage medium having program code embodied therewith, the program code executable by a processor to:
select a first imaging study in a worklist graphical user interface (GUI) in response to a first user input, wherein the first imaging study corresponds to at least one anatomical structure;
automatically identify, in a clinical reporting application (CRA) GUI, at least one vessel diagram corresponding to the at least one anatomical structure of the first imaging study based on the DICOM data associated with the first imaging study;
select a first vessel diagram of the automatically identified at least one vessel diagram in response to a second user input in the CRA GUI;
display, in the CRA GUI, the first vessel diagram with a set of predefined annotation tools;
determine if at least one characteristic of the at least one anatomical structure meets pre-determined criteria based on the DICOM data;
when the at least one characteristic of the at least one anatomical structure meets the pre-determined criteria, automatically incorporate at least one annotation with the first vessel diagram using at least one of the predefined annotation tools; and
store, in a memory of the PACS, the first vessel diagram with the at least one annotation.
